(19) United States
(12) Patent Application Publication
(10) Pub. No.: US 2011/0028559 A1
(43) Pub. Date: Feb. 3, 2011

(54) SUBSTITUTED FLUOROETHYL UREAS AS ALPHA 2 ADRENERGIC AGENTS

(76) Inventors: Wenkui k. Fang, Irvine, CA (US); Ken Chow, Newport Coast, CA (US); Daniel W. Gil, Corona Del Mar, CA (US); Evelyn G. Corpuz, Irvine, CA (US); Michael E. Garst, Newport Beach, CA (US)

Correspondence Address: ALLERGAN, INC., 2525 DUPONT DRIVE, T2-7H, IRVINE, CA, 92612-1599, US

(21) Appl. No.: 12/667,970

(22) PCT Filed: Jun. 20, 2008
(86) PCT No.: PCT/US08/67748

§ 371 Date: Oct. 22, 2010

Related U.S. Application Data
(60) Provisional application No. 60/948,289, filed on Jul. 6, 2007.

(51) Int. Cl. A61K 31/17; C07C 275/26; A61P 29/00

(52) U.S. Cl. 514/588; 564/57

(57) ABSTRACT

Treating pain in mammals with a compound having the formula (I) is disclosed herein.

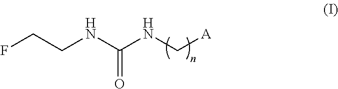

### CROSS-REFERENCE

This application claims the benefit of U.S. Application Ser. No. 60/948,389, filed Jul. 6, 2007, which is hereby incorporated by reference in its entirety.

### DESCRIPTION OF THE INVENTION

Compounds are disclosed herein having the formula

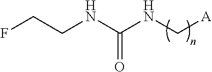

wherein n is 0 or 1; and
A is cyclohexyl having 0, 1, 2, 3, or 4 substituents
wherein the substituents each independently consist of: a moiety consisting of from 0 to 8 carbon atoms, 0 or 1 nitrogen atoms, 0 or 1 oxygen atoms, 0 or 1 sulfur atoms, 0 to 3 fluorine atoms, and from 0 to 17 hydrogen atoms; F; Cl; Br; or I.

These compounds are useful for treating pain in a mammal

One embodiment is a method of treating pain comprising administering a compound disclosed herein.

Another embodiment is use of a compound disclosed herein in the manufacture of a medicament for the treatment of pain.

In one embodiment the pain is chronic pain. “Chronic pain is pain that persists or recurs for [greater than] 3 months, persists [for greater than] 1 month after resolution of an acute tissue injury, or accompanies a nonhealing lesion.” (Online Merck Manual, Eighteenth Edition.)

In another embodiment the pain is neuropathic pain. “Neuropathic pain results from damage to or dysfunction of the peripheral or central nervous system.” (Online Merck Manual, Eighteenth Edition.)

In another embodiment the pain is visceral pain. “Visceral pain comes from the abdominal viscera, which are innervated by autonomic nerve fibers and respond mainly to the sensations of distention and muscular contraction.” (Online Merck Manual, Eighteenth Edition.)

In another embodiment the pain is allodynia. Allodynia is “pain due to a normoxious stimulus.” (Online Merck Manual, Eighteenth Edition.)

In another embodiment the pain is associated with muscle spasticity.

In another embodiment the pain is associated with diarrhea.

For the purposes of this disclosure, “treat,” “treating,” or “treatment” refers to the diagnosis, cure, mitigation, treatment, or prevention of disease or other undesirable condition. Other indications that the compounds can be used to treat include hypertension, congestive heart failure, asthma, depression, glaucoma, elevated intraocular pressure, ischemic neuropathies, optic neuropathy, corneal pain, headache pain, migraine, cancer pain, back pain, irritable bowel syndrome pain, muscle pain, pain associated with diabetic neuropathy, the treatment of diabetic retinopathy, stroke, drug dependence, withdrawal symptoms, obsessive compulsive disorder, obesity, insulin resistance, diarrhea, diuresis, nasal congestions, spasticity, attention deficit disorder, psychoses, Crohn's disease, gastritis, Alzheimer's disease, Parkinson's disease, amyotrophic lateral sclerosis, and combinations thereof

Unless otherwise indicated, reference to a compound should be construed broadly to include pharmaceutically acceptable salts, prodrugs, tautomers, alternate solid forms, and non-covalent complexes of a chemical entity of the depicted structure or chemical name.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt is a chemical species having an ionic form of the compound, such as a conjugate acid or base, associated with a corresponding amount of counter-ions. Salts can form from or incorporate one or more deprotonated acidic groups (e.g. carboxylic acids), one or more protonated basic groups (e.g. amines), or both (e.g. zwitterions).

A prodrug is a compound which is converted to a therapeutically active compound after administration to an animal or human. For example, conversion may occur by hydrolysis of an ester group or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in rapid equilibrium with one another. For example, they may include a transfer of a proton, hydrogen atom, or hydride ion. For example, the structures herein are intended to include, but are not limited to, the tautomeric forms shown below.

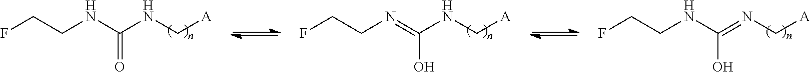

Unless stereochemistry is explicitly depicted, a structure is intended to include any possible stereoisomer, both pure or in any possible isomeric mixture.

Alternate solid forms are different solid forms than those that may result from practicing the procedures described herein. For example, alternate solid forms may be polymorphs, different kinds of amorphous solid forms, glasses, and the like.

Non-covalent complexes are complexes that may form between the compound and one or more additional chemical species that do not involve a covalent bonding interaction between the compound and the additional chemical species. They may or may not have a specific ratio between the compound and the additional chemical species. Examples might include solvates, hydrates, charge transfer complexes, and the like.

Because n is 0 or 1, the following compounds are possible.

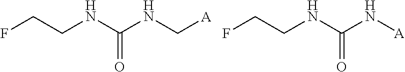

The substituents each independently consist of: a moiety consisting of from 0 to 8 carbon atoms, 0 or 1 nitrogen atoms, 0 or 1 oxygen atoms, 0 or 1 sulfur atoms, 0 to 3 fluorine atoms, and from 0 to 17 hydrogen atoms; F; Cl; Br; or I.

Subject to the constraints described herein (e.g. limits on the number of atoms for a substituent), examples of substituents include, but are not limited to:

Hydrocarbyl, meaning a moiety consisting of carbon and hydrogen only, including, but not limited to:
alkyl, meaning hydrocarbyl having no double or triple bonds, including, but not limited to:

linear alkyl, e.g. methyl, ethyl, n-propyl, n-butyl, n-pentyl, n-hexyl, etc.,

branched alkyl, e.g. iso-propyl, t-butyl and other branched butyl isomers, branched pentyl isomers, etc.,

cycloalkyl, e.g. cyclopropyl, cyclobutyl, cyclopentyl, cyclohexyl, etc.,

combinations of linear, branched, and/or cycloalkyl;

alkenyl, e.g. hydrocarbyl having 1 or more double bonds, including linear, branched, or cycloalkenyl

alkynyl, e.g. hydrocarbyl having 1 or more triple bonds, including linear, branched, or cycloalkenyl;

combinations of alkyl, alkenyl, and/or alkynyl

alkyl-CN, such as —CH₂—CN, —(CH₂)₂—CN; —(CH₂)₃—CN, and the like;

hydroxyalkyl, i.e. alkyl-OH, such as hydroxymethyl, hydroxyethyl, and the like;

ether substituents, including —O-alkyl, alkyl-O-alkyl, and the like;

thioether substituents, including —S-alkyl, alkyl-S-alkyl, and the like;

amine substituents, including —NH₂, —NH-alkyl, —N-alkyl¹alkyl² (i.e., alkyl¹ and alkyl² are the same or different, and both are attached to N), alkyl-NH₂, alkyl-NH-alkyl, alkyl-N-alkyl¹alkyl², and the like;

aminoalkyl, meaning alkyl-amine, such as aminomethyl (—CH₂-amine), aminoethyl, and the like;

ester substituents, including —CO₂-alkyl, —CO₂-phenyl, etc.;

other carbonyl substituents, including aldehydes; ketones, such as acyl (i.e.

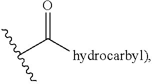

and the like; in particular, acetyl, propionyl, and benzoyl substituents are contemplated;

phenyl or substituted phenyl;

fluorocarbons or hydrofluorocarbons such as —CF₃, —CH₂CF₃, etc.; and

—CN;

combinations of the above are also possible, subject to the constraints defined;

Alternatively, a substituent may be —F, —Cl, —Br, or —I.

In particular, alkyl having from 1 to 8 carbon atoms is contemplated as a substituent.

Alternatively, alkyl having from 1 to 4 carbon atoms is contemplated;

Substituents must be sufficiently stable to be stored in a bottle at room temperature under a normal atmosphere for at least 12 hours, or stable enough to be useful for any purpose disclosed herein.

If a substituent is a salt, for example of a carboxylic acid or an amine, the counter-ion of said salt, i.e. the ion that is not covalently bonded to the remainder of the molecule is not counted for the purposes of determining the number of atoms in a substituent. Thus, for example, the salt —CO₂⁻Na⁺ is a stable substituent consisting of 1 carbon atom and 2 oxygen atoms, i.e. sodium is not counted. In another example, the salt —NH(Me)₂⁺Cl⁻ is a stable substituent consisting of 1 nitrogen atom, 2 carbon atoms, and 7 hydrogen atoms, i.e. chlorine is not counted.

In one embodiment the substituents are independently C₁₋₄ alkyl, C₁₋₃ alkoxy, F, Cl, Br, I, or NH₂.

Use of the notation “C_{x-y}” means the moiety has from x to y carbon atoms. For example, C₁₋₆ alkyl means alkyl having from 1 to 6 carbon atoms, or C₁₋₆ hydrocarbyl means hydrocarbyl having from 1 to 6 carbon atoms.

Another embodiment is a compound having the formula

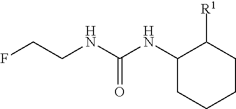

wherein R¹ is alkyl having from 1 to 6 carbon atoms.

In another embodiment, R¹ is n-propyl, i.e. —CH₂CH₂CH₃.

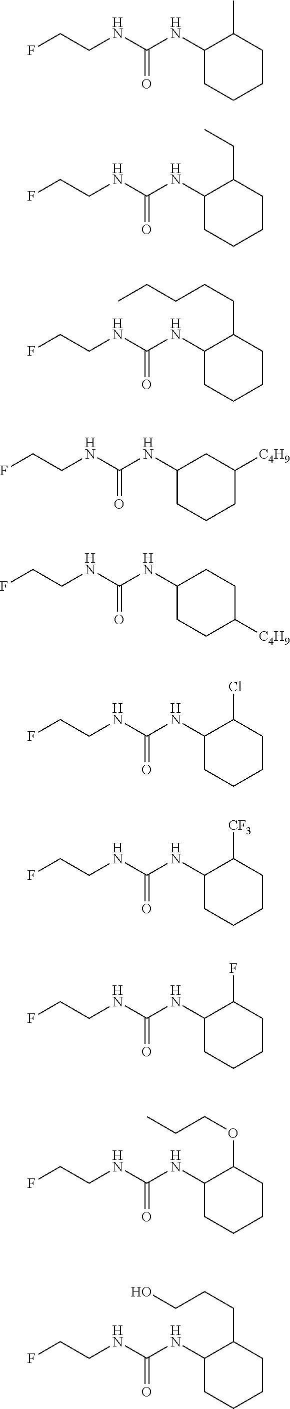

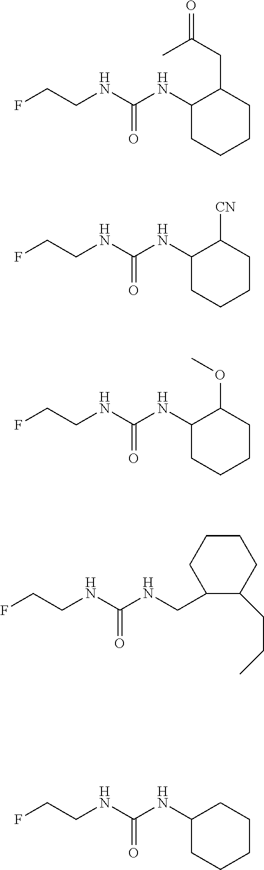

### Biological Data

U.S. Provisional Patent Application Ser. No. 60/911,442, filed Apr. 12, 2007, describes the procedure used to obtain the data in the table below. That procedure is incorporated by reference herein.

| | Alpha 2A | | Alpha 2B | | Alpha 2C | |
|---|---|---|---|---|---|---|
| Compound | EC₅₀ | IA | EC₅₀ | IA | EC₅₀ | IA |
| 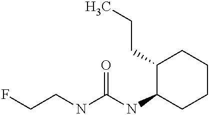 | 1475 | 0.4 | 1739 | 0.84 | not active | |

### Synthesis of 1-(2-fluoro-ethyl)-3-(2R-propyl-1R-cyclohexyl)-urea

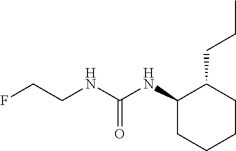

1-(2-Fluoro-ethyl)-3-(2R-propyl-1R-cyclohexyl)-urea: The title compound was obtained from 2R-propyl-1R-cyclohexylamine (1.40 g, 10.00 mmol), diimidazole carbonyl (1.70 g, 10.48 mmol), fluoroethyl amine hydrochloride (1.00 g, 90% purity, 9.05 mmol) and diisopropylethyl amine (3.60 mL, 20.67 mmol) according to the protocols as outlined in general procedure A, incorporated by reference, of U.S. 60/911,442, filed Apr. 12, 2007. Spectroscopic data: ¹H NMR (300 MHz, DMSO-d₆) δ ppm 0.76-0.88 (m, 2H) 1.21 (s, 2H) 1.36 (s, 2H) 3.57 (s, 2H) 4.51 (s, 4H) 4.66 (s, 2H) 7.19 (s, 4H) 7.29 (s, 2H) 7.43 (s, 4H) 8.75 (s, 2H).

Methods of formulating these compounds are well known in the art. For example, U.S. Pat. No. 7,141,597 (especially column 10, line 27 to column 14, line 47) contains information that may be used for general guidance. Similar relevant information is also available in numerous other sources. The biological activity of the compounds disclosed herein (e.g. Table 1) may be used for additional general guidance on dosage, depending on the particular use of a compound.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the claims.

What is claimed is:

1. A method of treating pain comprising administering a compound to a mammal in need thereof, said compound having the formula 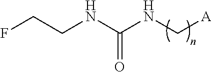
wherein n is 0 or 1; and
A is cyclohexyl having 0, 1, 2, 3, or 4 substituents
wherein the substituents each independently consist of: a moiety consisting of from 0 to 8 carbon atoms, 0 or 1 nitrogen atoms, 0 or 1 oxygen atoms, 0 or 1 sulfur atoms, 0 to 3 fluorine atoms, and from 0 to 22 hydrogen atoms; F; Cl; Br; or I.

2. The method of claim 1 wherein the pain is chronic pain.

3. The method of claim 1 wherein the pain is neuropathic pain.

4. The method of claim 1 wherein the pain is visceral pain.

5. The method of claim 1 wherein the pain is allodynia.

6. The method of claim 1 wherein the pain is associated with muscle spasticity.

7. The method of claim 1 wherein the pain is associated with diarrhea.

8. A compound having the formula 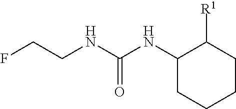
wherein R¹ is alkyl having from 1 to 6 carbon atoms.

9. The compound of claim 8 wherein R¹ is n-propyl.

10. The compound of claim 8 wherein the compound may be used to treat hypertension, congestive heart failure, asthma, depression, glaucoma, elevated intraocular pressure, ischemic neuropathies, optic neuropathy, corneal pain, headache pain, migraine, cancer pain, back pain, irritable bowel syndrome pain, muscle pain, pain associated with diabetic neuropathy, the treatment of diabetic retinopathy, stroke, drug dependence, withdrawal symptoms, obsessive compulsive disorder, obesity, insulin resistance, diarrhea, diuresis, nasal congestions, spasticity, attention deficit disorder, psychoses, Crohn's disease, gastritis, Alzheimer's disease, Parkinson's disease, amyotrophic lateral sclerosis, and combinations thereof.

11. A compound having the formula 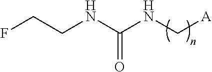
wherein n is 0 or 1; and
A is cyclohexyl having 0, 1, 2, 3, or 4 substituents
wherein the substituents each independently consist of: a moiety consisting of from 0 to 8 carbon atoms, 0 or 1 nitrogen atoms, 0 or 1 oxygen atoms, 0 or 1 sulfur atoms, 0 to 3 fluorine atoms, and from 0 to 22 hydrogen atoms; F; Cl; Br; or I.

12. The compound of claim 11 wherein the compound may be used to treat hypertension, congestive heart failure, asthma, depression, glaucoma, elevated intraocular pressure, ischemic neuropathies, optic neuropathy, corneal pain, headache pain, migraine, cancer pain, back pain, irritable bowel syndrome pain, muscle pain, pain associated with diabetic neuropathy, the treatment of diabetic retinopathy, stroke, drug dependence, withdrawal symptoms, obsessive compulsive disorder, obesity, insulin resistance, diarrhea, diuresis, nasal congestions, spasticity, attention deficit disorder, psychoses, Crohn's disease, gastritis, Alzheimer's disease, Parkinson's disease, amyotrophic lateral sclerosis, and combinations thereof.
